(19) United States
(12) Patent Application Publication
(10) Pub. No.: US 2009/0078030 A1
(43) Pub. Date: Mar. 26, 2009

(54) Test Sensor With a Fluid Chamber Opening

(76) Inventor: Sung-Kwon Jung, Granger, IN (US)

Correspondence Address: NIXON PEABODY LLP, 161 N. CLARK STREET, 48TH FLOOR, CHICAGO, IL, 60601, US

(21) Appl. No.: 11/989,342

(22) PCT Filed: Aug. 28, 2006
(86) PCT No.: PCT/US2006/033678

§ 371 Date: Jan. 24, 2008

Related U.S. Application Data
(60) Provisional application No. 60/712,484, filed on Aug. 30, 2005.

(51) Int. Cl. G01N 33/00; G01N 27/26; G01N 21/00

(52) U.S. Cl. 73/61.41; 204/400; 205/775; 356/72

(57) ABSTRACT

A test sensor adapted to assist in determining the analyte concentration in a fluid sample comprises a lid and a base. The lid has an upper lid surface and a lower lid surface. The lid has a first lid end, a second lid end, a first lid side, and a second lid side. The base has an upper base surface and a lower base surface. The base further has a first base end, a second base end, a first base side, and a second base side. The lid and base are attached such that a fluid chamber is formed between a portion of the lower lid surface and the upper base surface at or near the first lid end and the first base end. The fluid chamber is adapted to receive the fluid sample between at least the first base side and the first lid side, between the first lid end and the first lid end, or the combination thereof.

### FIELD OF THE INVENTION

The present invention generally relates to a test sensor. More specifically, the present invention generally relates to a test sensor with a fluid chamber that is adapted to receive fluid.

### BACKGROUND OF THE INVENTION

The quantitative determination of analytes in body fluids is of great importance in the diagnoses and maintenance of certain physiological abnormalities. For example, lactate, cholesterol and bilirubin should be monitored in certain individuals. In particular, it is important that diabetic individuals frequently check the glucose level in their body fluids to regulate the glucose intake in their diets. The results of such tests can be used to determine what, if any, insulin or other medication needs to be administered. In one type of blood-glucose testing system, test sensors are used to test a sample of blood.

The test sensor is adapted to receive fluid (e.g., blood) from a user. Existing test sensors differ in the manner in which they receive fluids. In one existing test sensor, a channel is formed between a generally U-shaped spacer and is adapted to receive blood from a user. A user then places blood from, for example, his/her finger into the channel. It has been observed that users may “abuse” such a test sensor by jamming the tip of the test sensor into the individual's finger, which results in the channel being temporarily blocked. Such temporary blockage can potentially lead to a biased reading. Additionally, in some existing test sensors, it is difficult to position the fluid sample within the channel.

Therefore, it would be desirable to have a test sensor that would (a) reduce or eliminate such a biased reading caused by such user action and/or (b) reduce the difficulty in properly positioning the fluid in the test sensor.

### SUMMARY OF THE INVENTION

According to one embodiment, a test sensor, which is adapted to assist in determining the concentration of an analyte in a fluid sample, comprises a lid and a base. The lid has an upper lid surface and a lower lid surface. The lid has a first lid end, a second lid end, a first lid side, and a second lid side. The base has an upper base surface and a lower base surface. The base further has a first base end, a second base end, a first base side, and a second base side. The lid and base are attached such that a fluid chamber is formed between a portion of the lower lid surface and the upper base surface at or near the first lid end and the first base end. The fluid chamber is adapted to receive the fluid sample between at least the first base side and the first lid side, between the first lid end and the first lid end, or the combination thereof.

According to another embodiment, a test sensor, which is adapted to assist in determining the concentration of an analyte in a fluid sample, comprises a lid, a base and a spacer. The lid has an upper lid surface and a lower lid surface. The lid has a first lid end, a second lid end, a first lid side and a second lid side. The base has an upper base surface and a lower base surface. The base further has a first base end, a second base end, a first base side and a second base side. The spacer is located between and attached to the lid and the base. The lid, base and spacer are positioned such that a fluid chamber is formed between a portion of the lower lid surface and the upper base surface at or near the first lid end and the first base end. The fluid chamber is adapted to receive the fluid sample between at least the first base side and the first lid side, between the first base end and the first lid end, or the combination thereof.

According to one method, an analyte concentration of a fluid sample is determined. A test sensor is provided having a lid and a base. The lid has an upper lid surface and a lower lid surface. The lid has a first lid end, a second lid end, a first lid side, and a second lid side. The base has an upper base surface and a lower base surface. The base further has a first base end, a second base end, a first base side, and a second base side. The lid and base are attached such that a fluid chamber is formed between a portion of the lower lid surface and the upper base surface at or near the first lid end and the first base end. The fluid sample is placed in the fluid chamber. The fluid chamber is adapted to receive the fluid sample between at least the first base side and the first lid side, between the first lid end and the first lid end, or the combination thereof. The analyte concentration of the sample is determined.

### BRIEF DESCRIPTION OF THE DRAWINGS

FIG. 1 is a top view of a base to be used in forming a test sensor according to one embodiment.

FIG. 2 is a top view of a lid to be used in forming a test sensor according to one embodiment.

FIG. 3a is a top view of the test sensor using the base of FIG. 1, the lid of FIG. 2 and an adhesive according to one embodiment.

FIG. 3b is a side view of the test sensor of FIG. 3a.

FIG. 3c is an enlarged view of the generally circular region FIG. 3c in FIG. 3b.

FIG. 3d is another side view of the test sensor of FIG. 3a.

FIG. 3e is an enlarged view of the generally circular region FIG. 3e in FIG. 3d.

FIG. 4a is a side view of the test sensor using the base of FIG. 1, the lid of FIG. 2 and a spacer according to one embodiment.

FIG. 4b is an enlarged view of the generally circular region FIG. 4b in FIG. 4a.

FIG. 4c is another side view of the test sensor of FIG. 4a.

FIG. 4d is an enlarged view of the generally circular region FIG. 4d in FIG. 4c.

### DETAILED DESCRIPTION OF ILLUSTRATED EMBODIMENTS

The present invention is directed to an improved test sensor that is adapted to assist in determining the analyte concentration of an analyte in a fluid. In one embodiment, a test sensor is adapted to receive a fluid sample and is analyzed using an instrument or meter. Analytes that may be measured include glucose, lipid profiles (e.g., cholesterol, triglycerides, LDL and HDL), microalbumin, hemoglobin A_{1C}, fructose, lactate, or bilirubin. It is contemplated that other analyte concentrations may be determined. The analytes may be in, for example, a whole blood sample, a blood serum sample, a blood plasma sample, other body fluids like ISF (interstitial fluid) and urine, and non-body fluids. As used within this application, the term “concentration” refers to an analyte concentration, activity (e.g., enzymes and electrolytes), titers (e.g., antibodies), or any other measure concentration used to measure the desired analyte.

The test sensors include at least a base and a lid. The base and lid may be made from a variety of materials such as polymeric materials. Non-limiting examples of polymeric materials that may be used to form the base and lid include polycarbonate, polyethylene terephthalate (PET), polyethylene naphthalate (PEN), polyimide and combinations thereof. As will be discussed below, the test sensors may include an additional layer such as a spacer. Thus, in one embodiment, the test sensor includes a base, spacer and lid. The test sensors may be formed by a variety of methods including printing (e.g., screen-printing), coating (e.g., reverse roll), vapor deposition, sputtering, and electrochemical deposition.

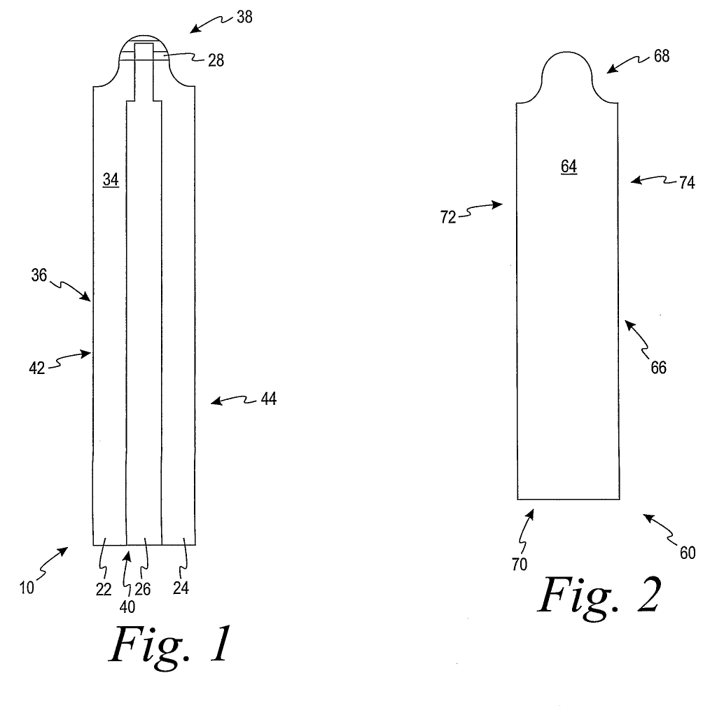

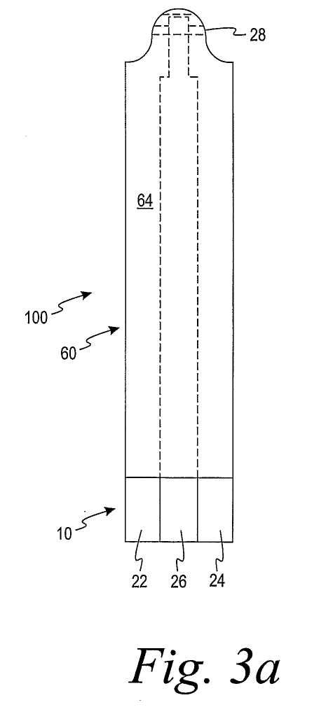

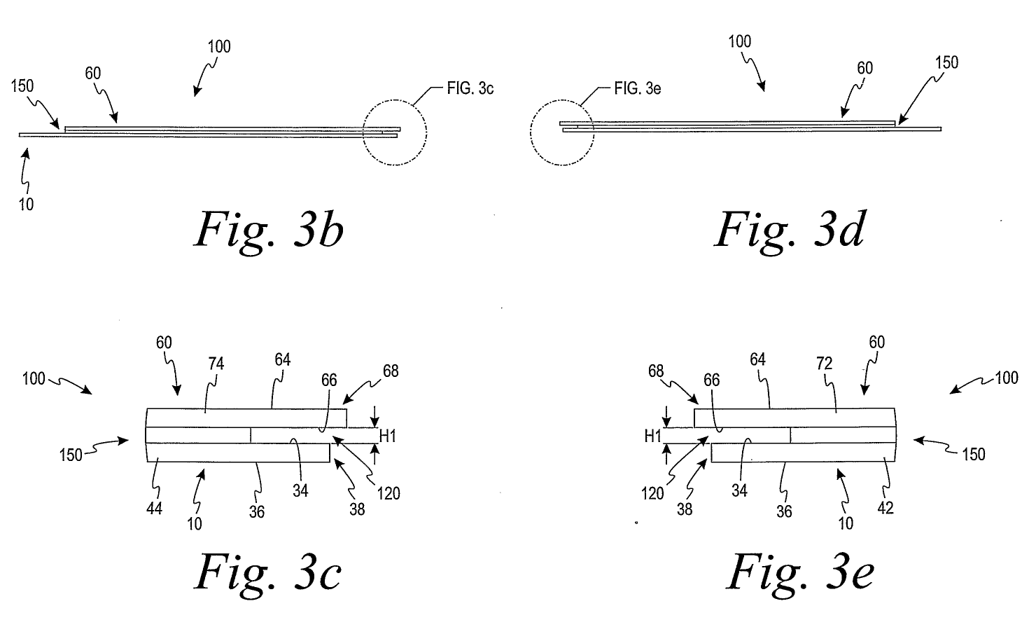

In one embodiment, the test sensor is an electrochemical test sensor. One non-limiting example of a test sensor (test sensor 100) is shown in FIGS. 3a-3e. The test sensor 100 of FIGS. 3a-3e is formed using a base 10 of FIG. 1 and a lid 60 of FIG. 2. The test sensor 100 of FIGS. 3a-3e includes the base 10, the lid 60 and an adhesive 150. When the base 10 and the lid 60 are attached together, a fluid chamber 120 is formed. The fluid chamber 120 provides a flow path for introducing the sample into the test sensor 100 and eventually contacting the electrodes, as will be discussed below.

Referring back to FIG. 1, the base 10 is shown that includes a plurality of electrodes 22, 24, 26 and a fluid-receiving area 28 that contains an enzyme. The enzyme is selected to react with the desired analyte or analytes to be tested so as to assist in determining an analyte concentration of a fluid sample. The fluid-receiving area 28 includes a reagent for converting an analyte of interest (e.g., glucose) in a fluid test sample (e.g., blood) into a chemical species that is electrochemically measurable, in terms of the electrical current it produces, by the components of the electrode pattern. The reagent typically contains an enzyme such as, for example, glucose oxidase, which reacts with the analyte and with an electron acceptor such as a ferricyanide salt to produce an electrochemically measurable species that can be detected by the electrodes. It is contemplated that other enzymes may be used to react with glucose such as glucose dehydrogenase. If the concentration of another analyte is to be determined, an appropriate enzyme is selected to react with the analyte.

The fluid-receiving area 28 may comprise a polymer, an enzyme, and an electron acceptor. The fluid-receiving area 28 may further include a mediator that is an electron acceptor and assists in generating a current that corresponds to the analyte concentration. If the enzyme is glucose oxidase, then a mediator (e.g., potassium ferricyanide) may be included. The fluid-receiving area 28 also may include additional ingredients such as a buffer and a surfactant in some embodiments.

The plurality of electrodes includes counter electrodes 22, 24 and a working electrode 26 in this embodiment. In one embodiment, an analyte concentration is only reported if the tested fluid contacts both of the counter electrodes and, thus, the test sensor in this embodiment has underfill protection. In another embodiment, the plurality of electrodes includes one counter electrode and two working electrodes. In this embodiment, the analyte concentration of one working electrode should be the same or generally correspond to the other analyte concentration of the other working electrodes to ensure that the sample size is sufficient. Thus, this embodiment also has underfill protection.

It is contemplated that more or less electrodes may be formed in the base that is used in forming the test sensor. For example, in other embodiments, the test sensor may include exactly two electrodes or at least four electrodes. The exactly two electrodes may be a working and counter electrode in which an electrochemically created current flows when these electrodes are electrically connected and potential created between them.

The flow of electrons created by the enzymatic reaction flows through the working electrode to a meter that measures the magnitude of the current flow. The counter electrode provides a fixed potential against which the working electrode is controlled. The counter electrode may also be used to complete the electrical circuit. As shown in this embodiment, the detection electrode may be an electrode that detects an underfill condition. It is contemplated that other electrodes may be used such as a hematocrit electrode that assists in correcting for the bias that occurs with selected hematocrit concentrations.

The electrodes may be formed on the base by a variety of methods such as, for example, printing onto the base. The electrodes are formed of conductive materials such as, for example, metallic materials (e.g., gold, platinum, palladium, rhodium, ruthenium, or combinations thereof) or carbon.

The electrodes may be defined by a laser using a mask. For example, the plurality of electrodes 22, 24, 26 may be defined by using a mask and a laser such as, for example, an Excimer laser or a carbon dioxide-based laser. One example of a mask is a chrome-on-glass mask in which the beam of light is only allowed to pass through selected areas. According to another method, the plurality of electrodes may be defined with a laser using direct writing of the lines. In this method, the laser beam of light is moved so as to define the plurality of electrodes. Lasers that produce a beam of energy capable of removing a layer and that can be moved to form a pattern may be used in this method. Non-limiting examples of such lasers are carbon dioxide-based lasers and yttrium-based lasers such as yttrium aluminum garnet (YAG) lasers.

It is contemplated that the plurality of electrodes may be defined by other methods such as, for example, printing (e.g., screen-printing), coating (e.g., reverse roll), vapor deposition, sputtering, and electrochemical deposition.

The base 10 of FIG. 1 includes an upper base surface 34 and a lower base surface 36. The base 10 includes a first base end 38, a second base end 40, a first base side 42, and a second base side 44. The first base end 38 and the second base end 40 are located on opposing ends of the base 10. The first base side 42 and the second base side 44 are located on opposing sides of the base 10.

Similarly, the lid 60 of FIG. 2 includes an upper lid surface 64 and a lower lid surface 66. The lid 60 includes a first lid end 68, a second lid end 70, a first lid side 72, and a second lid side 74. The first lid end 68 and the second lid end 70 are located on opposing ends of the lid 60. The first lid side 72 and the second lid side 74 are located on opposing sides of the lid 60. The lower lid surface may be treated with surfactant to enhance the sample harvesting.

Examples of components, such as those mentioned above, used in forming electrochemical test sensors, including their operation, may be found in, for example, U.S. Pat. No. 6,531,040 B2.

It is contemplated that the test sensors may be other types of test sensors such as optical test sensors or calorimetric test sensors.

To form the test sensor 100 of FIGS. 3a-3e, the base 10 and the lid 60 are attached. In one embodiment, the base 10 is laminated to the lid 60 via the adhesive 150 to form the test sensor such as shown in FIGS. 3b-3e. It is contemplated that other materials may be used that have sticking properties such that the lid and the base remain attached.

The base 10 may be laminated to the lid 60 using, for example, a pressure-sensitive adhesive and/or a hot melt adhesive. Thus, the lamination between the base and the lid uses pressure, heat or a combination thereof. It is contemplated that other materials may be used to attach the base to the second surface.

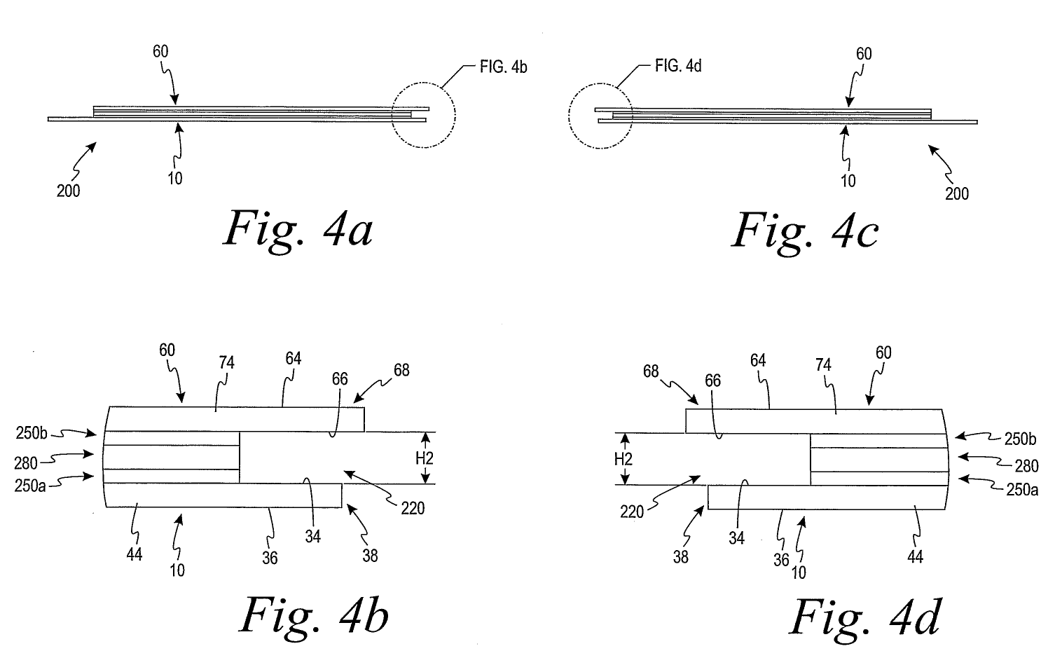

It is also contemplated that a spacer may be included in forming the test sensor such as will be discussed below with respect to the embodiment depicted in FIGS. 4a-4d. It is contemplated that the base and the lid may be heat-sealed to each other to form the test sensor. This may be accomplished using, for example, sonic welding.

After the base 10 and the lid 60 are attached, the fluid chamber 120 is formed between a portion of the lower lid surface 66 and the upper base surface 34 at or near the first lid end 68 and the first base end 38. The fluid chamber 120 is adapted to receive the fluid between at least one of the base sides and one of the lid sides, between the first base end 38 and the first lid end 68, or the combination thereof. Thus, in this embodiment, the test sensor 100 may be filled from (a) at least one side, (b) one end, or (b) from the side and the end at the same time. By having a test sensor with an adjoining side and end being adapted to receive fluid, the test sensor more easily receives the fluid from a user and is more tolerant to users who jam the tip of the sensor into his/her finger.

As shown in FIGS. 3b-3e, the test sensor 100 may be filled from (a) either of the sides, (b) one end or (c) a combination of the end and one or more of the sides. Thus, fluid chamber 120 is adapted to receive the fluid between at least the first base side 42 and the first lid side 72 (FIG. 3e), between the first base end 38 and the first lid end 68 (FIGS. 3c, 3e), the second base side 44 and the second lid side 74 (FIG. 3c), or any combination thereof.

The fluid chamber 120 as shown in FIGS. 3c and 3e has a height H1 that is generally from about 1 to about 10 mils. More specifically, the fluid chamber 120 as shown in FIGS. 3c and 3e has a height H1 that is generally from about 3 to about 7 mils. It is desirable for the height H1 to be able to receive the fluid (e.g., blood) from a user while still maintaining the blood within the confines of the fluid chamber 120.

Another example of a test sensor (test sensor 200) is shown in FIGS. 4a-4d. The test sensor 200 of FIGS. 4a-4d may be formed by using the base 10 of FIG. 1, the lid 60 of FIG. 2, and a spacer 280. The test sensor 200 of FIGS. 4a-4d includes the base 10, the lid 60, the spacer 280 and a fluid chamber 220 is formed when the base, spacer and the lid are attached together.

To form the test sensor 200 of FIGS. 4a-4d, the base 10, the spacer 280, and the lid 60 are attached. In one embodiment, the base 10 and the spacer 280 are attached via an adhesive 250a and the spacer 280 and the lid 60 are attached via an adhesive 250b.

The base 10 may be laminated to the spacer 280 using, for example, a pressure-sensitive adhesive and/or a hot melt adhesive. Thus, the lamination between the base and the spacer uses pressure, heat or a combination thereof. It is contemplated that other materials may be used to attach the base to the spacer. Similarly, the lid 60 and the spacer 280 may be attached using the same or a different adhesive than the adhesive used between the base 10 and the spacer 280.

It is contemplated that the lid and spacer may be attached by other methods such as heat sealing. Similarly, the base and the spacer may be attached by other methods such as heat sealing. Thus, in this embodiment, the test sensor would include a base, a spacer and a lid without an adhesive layer. The heat sealing may be accomplished by, for example, sonic welding. For example, the spacer may be made of a lower melting temperature material than the lid and the base.

In another embodiment, the lid or base may be heat-sealed to the spacer with the remaining one of the lid and base being adhesively attached to the spacer. For example, the lid and spacer may be heated sealed and the base is attached to the spacer via an adhesive layer. This would be the same as shown in FIGS. 4a-4d with the adhesive layer 250b being removed.

According to another embodiment, a spacer-lid combination is used in which the spacer and lid have been previously attached before being attached to the base. According to a further embodiment, a spacer-base combination is used in which the spacer and the base have been previously attached being attached to the lid.

After the base 10 and the lid 60 are attached, a fluid chamber 220 is formed between a portion of the lower lid surface 66 and the upper base surface 34 at or near the first lid end 68 and the first base end 38. The fluid chamber 220 is adapted to receive the fluid between one of the base sides and one of the lid sides, between the first base end 38 and the first lid end 68, or the combination thereof. Thus, in this embodiment, the test sensor 200 may be filled from at least one side, filled from the end, or filled from the side and the end. As shown in FIGS. 4a-4d, the test sensor 220 may be filled from one or both of the sides, as well as the end. Thus, fluid chamber 220 is adapted to receive the fluid between at least the first base side 42 and the first lid side 72 (FIG. 4d), between the first base end 38 and the first lid end 68 (FIGS. 4b, 4d), the second base side 44 and the second lid side 74 (FIG. 4b), or any combination thereof.

The fluid chamber 220 as shown in FIGS. 4b, 4d has a height H2 that is generally from about 1 to about 10 mils. More specifically, the fluid chamber 220 as shown in FIGS. 4b, 4d has a height H2 that is generally from about 3 to about 7 mils. It is desirable for the height H2 to be able to receive the fluid (e.g., blood) from a user while still maintaining the blood within the confines of the fluid chamber 220.

### Alternative Embodiment A

A test sensor adapted to assist in determining the concentration of an analyte in a fluid sample, the test sensor comprising:

a lid having an upper lid surface and a lower lid surface, the lid having a first lid end, a second lid end, a first lid side, and a second lid side; and

a base having an upper base surface and a lower base surface, the base further having a first base end, a second base end, a first base side, and a second base side, the lid and base being attached such that a fluid chamber is formed between a portion of the lower lid surface and the upper base surface at or near the first lid end and the first base end, the fluid chamber being adapted to receive the fluid sample between at least the first base side and the first lid side, between the first lid end and the first lid end, or the combination thereof.

### Alternative Embodiment B

The test sensor of Alternative Embodiment A wherein the fluid chamber formed between the portion of the lower lid surface and the upper base surface has a height of from about 1 to about 10 mils.

### Alternative Embodiment C

The test sensor of Alternative Embodiment B wherein the fluid chamber formed between the portion of the lower lid surface and the upper base surface has a height of from about 3 to about 7 mils.

### Alternative Embodiment D

The test sensor of Alternative Embodiment A wherein the fluid chamber is further adapted to receive the fluid sample between the second base side and the second lid side.

### Alternative Embodiment E

The test sensor of Alternative Embodiment A wherein the base is laminated to the lid.

### Alternative Embodiment F

The test sensor of Alternative Embodiment A wherein the test sensor is an electrochemical test sensor and the base further includes a plurality of electrodes.

### Alternative Embodiment G

The test sensor of Alternative Embodiment A wherein the test sensor is an optical test sensor.

### Alternative Embodiment H

A test sensor adapted to assist in determining the concentration of an analyte in a fluid sample, the test sensor comprising:

a lid having an upper lid surface and a lower lid surface, the lid having a first lid end, a second lid end, a first lid side and a second lid side;

a base having an upper base surface and a lower base surface, the base further having a first base end, a second base end, a first base side and a second base side; and

a spacer being located between and attached to the lid and the base,

wherein the lid, base and spacer are positioned such that a fluid chamber is formed between a portion of the lower lid surface and the upper base surface at or near the first lid end and the first base end, the fluid chamber being adapted to receive the fluid sample between at least the first base side and the first lid side, between the first base end and the first lid end, or the combination thereof.

### Alternative Embodiment I

The test sensor of Alternative Embodiment H wherein the spacer is directly attached to at least one of the lid and the base.

### Alternative Embodiment J

The test sensor of Alternative Embodiment I wherein the spacer is directly attached to both the lid and the base.

### Alternative Embodiment K

The test sensor of Alternative Embodiment I further including a first adhesive and a second adhesive, the first adhesive being located between the lid and the spacer, the second adhesive being located between the base and the spacer.

### Alternative Embodiment L

The test sensor of Alternative Embodiment H wherein the fluid chamber formed between the portion of the lower lid surface and the upper base surface has a height of from about 1 to about 10 mils.

### Alternative Embodiment M

The test sensor of Alternative Embodiment L wherein the fluid chamber formed between the portion of the lower lid surface and the upper base surface has a height of from about 3 to about 7 mils.

### Alternative Embodiment N

The test sensor of Alternative Embodiment H wherein the fluid chamber is further adapted to receive the fluid sample between the second base side and the second lid side.

### Alternative Embodiment O

The test sensor of Alternative Embodiment H wherein the base is laminated to the lid.

### Alternative Embodiment P

The test sensor of Alternative Embodiment H wherein the test sensor is an electrochemical test sensor and the base further includes a plurality of electrodes.

### Alternative Embodiment Q

The test sensor of Alternative Embodiment H wherein the test sensor is an optical test sensor.

### Alternative Process R

A method of determining an analyte concentration of a fluid sample, the method comprising the acts of:

providing a test sensor having a lid and a base, the lid having an upper lid surface and a lower lid surface, the lid having a first lid end, a second lid end, a first lid side, and a second lid side, the base having an upper base surface and a lower base surface, the base further having a first base end, a second base end, a first base side, and a second base side, the lid and base being attached such that a fluid chamber is formed between a portion of the lower lid surface and the upper base surface at or near the first lid end and the first base end;

placing the fluid sample in the fluid chamber, the fluid chamber being adapted to receive the fluid sample between at least the first base side and the first lid side, between the first lid end and the first lid end, or the combination thereof; and

determining the analyte concentration of the sample.

### Alternative Process S

The method of Alternative Process R wherein the fluid chamber is further adapted to receive the fluid sample between the second base side and the second lid side.

### Alternative Process T

The method of Alternative Process R wherein the test sensor further includes a spacer, the spacer being located between the lid and the base.

### Alternative Process U

The method of Alternative Process R wherein the test sensor is an electrochemical test sensor and the base further includes a plurality of electrodes.

### Alternative Process V

The method of Alternative Process R wherein the test sensor is an optical test sensor.

While the present invention has been described with reference to one or more particular embodiments, those skilled in the art will recognize that many changes may be made thereto without departing from the spirit and scope of the present invention. Each of these embodiments, and obvious variations thereof, is contemplated as falling within the spirit and scope of the invention as defined by the appended claims.

1. A test sensor adapted to assist in determining the concentration of an analyte in a fluid sample, the test sensor comprising:
a lid having an upper lid surface and a lower lid surface, the lid having a first lid end, a second lid end, a first lid side, and a second lid side; and
a base having an upper base surface and a lower base surface, the base further having a first base end, a second base end, a first base side, and a second base side, the lid and base being attached such that a fluid chamber is formed between a portion of the lower lid surface and the upper base surface at or near the first lid end and the first base end, the fluid chamber being adapted to receive the fluid sample between at least the first base side and the first lid side, between the first lid end and the first lid end, or the combination thereof.

2. The test sensor of claim 1, wherein the fluid chamber formed between the portion of the lower lid surface and the upper base surface has a height of from about 1 to about 10 mils.

3. The test sensor of claim 2, wherein the fluid chamber formed between the portion of the lower lid surface and the upper base surface has a height of from about 3 to about 7 mils.

4. The test sensor of claim 1, wherein the fluid chamber is further adapted to receive the fluid sample between the second base side and the second lid side.

5. The test sensor of claim 1, wherein the base is laminated to the lid.

6. The test sensor of claim 1, wherein the test sensor is an electrochemical test sensor and the base further includes a plurality of electrodes.

7. The test sensor of claim 1, wherein the test sensor is an optical test sensor.

8. A test sensor adapted to assist in determining the concentration of an analyte in a fluid sample, the test sensor comprising:
a lid having an upper lid surface and a lower lid surface, the lid having a first lid end, a second lid end, a first lid side and a second lid side;
a base having an upper base surface and a lower base surface, the base further having a first base end, a second base end, a first base side and a second base side; and
a spacer being located between and attached to the lid and the base,
wherein the lid, base and spacer are positioned such that a fluid chamber is formed between a portion of the lower lid surface and the upper base surface at or near the first lid end and the first base end, the fluid chamber being adapted to receive the fluid sample between at least the first base side and the first lid side, between the first base end and the first lid end, or the combination thereof.

9. The test sensor of claim 8, wherein the spacer is directly attached to at least one of the lid and the base.

10. The test sensor of claim 9, wherein the spacer is directly attached to both the lid and the base.

11. The test sensor of claim 9, further including a first adhesive and a second adhesive, the first adhesive being located between the lid and the spacer, the second adhesive being located between the base and the spacer.

12. The test sensor of claim 8, wherein the fluid chamber formed between the portion of the lower lid surface and the upper base surface has a height of from about 1 to about 10 mils.

13. The test sensor of claim 12, wherein the fluid chamber formed between the portion of the lower lid surface and the upper base surface has a height of from about 3 to about 7 mils.

14. The test sensor of claim 8, wherein the fluid chamber is further adapted to receive the fluid sample between the second base side and the second lid side.

15. The test sensor of claim 8, wherein the base is laminated to the lid.

16. The test sensor of claim 8, wherein the test sensor is an electrochemical test sensor and the base further includes a plurality of electrodes.

17. The test sensor of claim 8, wherein the test sensor is an optical test sensor.

18. A method of determining an analyte concentration of a fluid sample, the method comprising the acts of:
providing a test sensor having a lid and a base, the lid having an upper lid surface and a lower lid surface, the lid having a first lid end, a second lid end, a first lid side, and a second lid side, the base having an upper base surface and a lower base surface, the base further having a first base end, a second base end, a first base side, and a second base side, the lid and base being attached such that a fluid chamber is formed between a portion of the lower lid surface and the upper base surface at or near the first lid end and the first base end;
placing the fluid sample in the fluid chamber, the fluid chamber being adapted to receive the fluid sample between at least the first base side and the first lid side, between the first lid end and the first lid end, or the combination thereof; and
determining the analyte concentration of the sample.

19. The method of claim 18, wherein the fluid chamber is further adapted to receive the fluid sample between the second base side and the second lid side.

20. The method of claim 18, wherein the test sensor further includes a spacer, the spacer being located between the lid and the base.

21. The method of claim 18, wherein the test sensor is an electrochemical test sensor and the base further includes a plurality of electrodes.

22. The method of claim 18, wherein the test sensor is an optical test sensor.
